(19) United States
(12) Patent Application Publication
(10) Pub. No.: US 2017/0191910 A1
(43) Pub. Date: Jul. 6, 2017

(54) METHOD, COMPUTING DEVICE AND SYSTEM FOR COLLECTING EXHALED BREATH

(71) Applicant: THE CLEVELAND CLINIC FOUNDATION, Cleveland, OH (US)

(72) Inventors: Daniel Laskowski, Cleveland, OH (US); Raed Dweik, Moreland Hills, OH (US)

(21) Appl. No.: 15/393,450

(22) Filed: Dec. 29, 2016

Related U.S. Application Data
(60) Provisional application No. 62/273,561, filed on Dec. 31, 2015.

(51) Int. Cl. G01N 1/40; A61B 5/00; A61B 5/08; G01N 1/22; A61B 5/091

(52) U.S. Cl. CPC G01N 1/40; G01N 1/22; A61B 5/091; A61B 5/082; A61B 5/742; G01N 2001/2244

(57) ABSTRACT

Various methods, computing devices, and systems for collecting breath from a subject are described. In one aspect, a method for collecting breath from a subject includes capturing a target sample from an exhaled breath of a subject having a target concentration of a target volatile organic compound (VOC). The target sample of the target VOC is captured from the non-alveolar volume fraction of the exhaled breath, and the exhaled breath represents the lung capacity of the subject. The target concentration of a target VOC can be, for example, an elevated or a lowered concentration of the target sample of exhaled breath relative to the remaining fractions of the exhaled breath, and can found in a particular fraction of the exhaled breath depending on the particular compound targeted for collection or analysis.

### RELATED APPLICATION

This patent application claims priority to Application Ser. No. 62/273,561 entitled “Method, Computing Device and System for Collecting Exhaled Breath” filed on Dec. 31, 2015, the entirety of which is incorporated by reference herein.

### TECHNICAL FIELD

The present invention relates generally to methods, computing devices and systems for collecting exhaled breath of a subject. More specifically, the present invention relates to methods, computing devices and systems for collecting exhaled breath of a subject for determining concentrations of volatile organic compounds in a breath sample of the subject.

### BACKGROUND

To date, the clinical use and determination of breath volatile organic compounds (VOCs) has proven to be a significant medical tool in the diagnosis and/or the detection of various diseases. The measuring of VOCs in exhaled breath is moving from research to clinical application. Different methods are used by various researchers to capture the exhaled gas for analysis. The chemical analysis of breath involves the measurement of very low concentrations of the large numbers of VOCs that can be found in the breath. The sensitivity of equipment measuring VOCs in the breath is in the order of parts per billion (ppb).

The analysis of breath provides insight to the composition of blood because many breath VOCs originate from the blood. That is, many VOCs enter the alveoli of the lungs by diffusion across the pulmonary alveolar membrane. This volume of breath is known as the “alveolar” portion of exhaled breath. Other VOCs and gases in the lungs do not originate from the blood and are present from the pharynx, trachea and bronchial cells that do not exchange compounds from the blood stream. This volume of breath is often referred to the “dead space”.

Current apparatus and methods of collecting and/or analyzing the breath of a patient or subject can be burdensome and/or provide inconsistent results of exhaled breath compositions and VOC concentrations for purposes of meaningful and reliable clinical application.

### SUMMARY

Various methods for collecting breath from a subject are described. In one aspect, a method for collecting breath from a subject includes capturing a target sample from an exhaled breath of a subject that contains an target concentration of a target volatile organic compound (VOC). The target sample of the target VOC is captured from the non-alveolar volume fraction of the exhaled breath, and where the exhaled breath represents the lung capacity of the subject. The target concentration can be, for example, an elevated concentration of the target VOC, or in another example, a low level concentration, depending on the target VOC. In another example the target sample of the target VOC is captured from a first about 80% volume fraction, and in another example from the volume fraction ranging from the first about 20% volume to the first about 80% volume fraction of exhaled breath.

In another example of the present invention a computing device or a non-transitory, machine-readable medium having machine-executable instructions, for example a computer, controller, etc., is configured to capture a target sample in the non-alveolar volume fraction of an exhaled breath of a subject, the target sample comprising an target concentration of a target volatile organic compound and where the exhaled breath represents the lung capacity of the subject. In another example the target sample of the target VOC is captured from a first about 80% volume fraction, and in another example from the volume fraction ranging from the first about 20% volume to the first about 80% volume fraction of exhaled breath.

In yet another example, a system includes a breath collection device and a computing device in communication with the breath collection device. The computing device comprises a non-transitory, machine-readable medium having machine-executable instructions, for example a computer, controller, etc., configured to capture a target sample in the non-alveolar volume fraction of an exhaled breath stream of a subject, the target sample comprising an target concentration of a target volatile organic compound, and where the exhaled breath represents substantially the lung capacity of the subject. In another example the target sample of the target VOC is captured from a first about 80% volume fraction, and in another example from the volume fraction ranging from the first about 20% volume to the first about 80% volume fraction of exhaled breath.

### BRIEF DESCRIPTION OF THE FIGURES

The example embodiments of the present invention can be understood with reference to the attached figures. The components in the figures are not necessarily drawn to scale. Also, in the figures, like reference numerals designate corresponding parts throughout the views.

FIG. 1 is a schematic representation of a system that includes a breath collection device and a computing device, according to an example of the present invention;

FIG. 2 is a schematic side-view illustration of an example of the breath collection device of the system of FIG. 1, according to an example of the present invention;

FIG. 3 is a flow chart illustrating a method of implementing the system of FIG. 1, according to an example of the present invention; and

FIG. 4 is a schematic representation of a system that includes a breath collection device, according to another example of the present invention.

### DETAILED DESCRIPTION

The various examples, methods, devices and systems of the present invention relate to exhaled breath fractionation. Known methods and devices for collecting breath for analysis have focused on the collection of breath and VOCs which have diffused from the blood and drawn specifically from the alveoli of the lungs which are represented by the tail-end of exhaled breath.

According to an aspect of the present invention various methods, devices and systems implementing methods of breath fractionation can identify endogenous and exogenous breath compounds in exhaled breath to properly diagnose patients. It is found herein that a target concentration of volatile organic compounds (VOCs) which correlate with various disease states, surprisingly, can be found in the non-alveolar fraction of exhaled breath, in another example in the first about 80% volume fraction of exhaled breath, in another example, in the first about 70% volume fraction of exhaled breath, and in another example, in the about 20% to about the 80% volume fraction of exhaled breath, in another example in the first about 20% to about 60% volume fraction, in another example in the first about 20% to about 40% volume fraction, in another example in the first about 40% to about 80% volume fraction, in another example in the first about 40% to about 60% volume fraction, and in another example in the first about 60% to about 80% volume fraction of the exhaled breath.

It is also found herein that the fraction of breath containing the target concentration that is an elevated concentration of a volatile organic compound can vary from volatile organic compound to volatile organic compound within the non-alveolar fraction of the exhaled breath rather than the “tail-end” volume fraction of the exhaled breath, the alveolar breath, which comes from deep within the lung closest to the blood stream. The fraction of breath containing the target concentration can be found to exist in any fraction of the exhaled breath depending upon the specific VOC. That is, an elevated or lowered level of concentration, for example can be found in the middle fraction that is between the dead space and alveolar fractions, rather than solely in the dead space or alveolar fractions, where molecules of the compound originating from an endogenous or exogenous source may have been expected to reside.

The terms “individual,” “subject,” and “patient” are used interchangeably herein irrespective of whether the subject has or is currently undergoing any form of treatment. As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Treatment of humans is of particular interest.

The term “volatile organic compound” or “VOC” refers to any compound of carbon, excluding carbon monoxide, and carbon dioxide.

The term “total lung capacity” refers to the total volume of gas contained in a subject's lungs and includes the volume of gas the subject can naturally exhale and the volume of gas that the subject cannot naturally exhale in a single breath. Gas that the subject cannot naturally exhale in a single breath could be, for example, the volume of gas that can be exhaled with the assistance of a machine or device. The total lung capacity will vary from patient to patient.

The terms “lung capacity” and “forced vital capacity” both refer to an established baseline volume in the lungs of the patient which is the maximum volume of gas the subject can naturally exhale in a single breath, and will vary from patient to patient. The lung capacity is less than a “total lung capacity” which includes the volume of gas that cannot be naturally exhaled.

The term “exhaled breath” refers to the volume of breath the subject naturally exhales in a single exhalation. An exhaled breath is less than the total lung capacity of the subject and exhaled breath is approximately equal to the lung capacity and the forced vital capacity of the patient.

The term “target sample” refers to a portion, or breath fraction, of the exhaled breath that includes a target concentration of a target VOC.

The term “target concentration” is the targeted concentration of the VOC to be captured. For example, the target concentration can be an elevated concentration or a lowered concentration of a target VOC relative to the concentration of the target VOC in the remaining fractions of the exhaled breath. The target concentration that is an elevated concentration of a target VOC can be at least one of the following: 1) a maximum number or “peak number” of molecules for the defined volume fraction of exhaled breath relative to the number of molecules in any of the remaining breath fractions of the exhaled breath; 2) a maximum average mean number of molecules for the defined volume fraction of gas that relative to the average mean number of molecules in any of the remaining breath fractions of the exhaled breath; and/or 3) a maximum median number of molecules, for a defined volume fraction of exhaled breath relative to the median number of molecules of the remaining breath fractions of the exhaled breath. In another example, a target concentration that is an lowered concentration can be at least one of the following: 1) a minimum number of molecules for the defined volume fraction of exhaled breath relative to the number of molecules of the remaining breath fractions of the exhaled breath; 2) a minimum average mean number of molecules for the defined volume fraction of exhaled breath relative to the average mean number of molecules of the remaining breath fractions of the exhaled breath; and/or 3) a minimum median number of molecules, for a defined volume fraction of exhaled breath relative to the median number of molecules of the remaining breath fractions of the exhaled breath. The target sample or breath fraction having an elevated VOC or a lowered VOC can vary from compound to compound.

The term “target sample size” refers to the volume of the target sample that is captured from the exhaled breath and is a smaller volume than the exhaled breath.

The term “alveolar breath” refers to the portion of exhaled breath from the deepest part of the lung and the tail-end of the breath exhaled by the subject or patient. As used herein it is the fraction of exhaled breath that follows and is greater than the first 80% by volume of the exhaled breath, in another example greater than the first 85% by volume of the exhaled breath, and in another example greater than the first 90% by volume of the exhaled breath, where the exhaled breath is equal to the lung capacity or the forced vital capacity of the subject.

The term “non-alveolar breath” refers to the portion of exhaled breath that precedes the tail-end of the exhaled breath and represents the first about 80% by volume of exhaled breath, in another example the first 85% by volume of exhaled breath, and in another example the first 90% by volume of exhaled breath, where the exhaled breath is equal to the lung capacity or the forced vital capacity of the subject.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. As used herein and in the appended claims, the singular forms “a”, “and”, and “the” include plural referents unless the context clearly dictates otherwise.

Unless otherwise indicated, all numbers expressing quantities used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

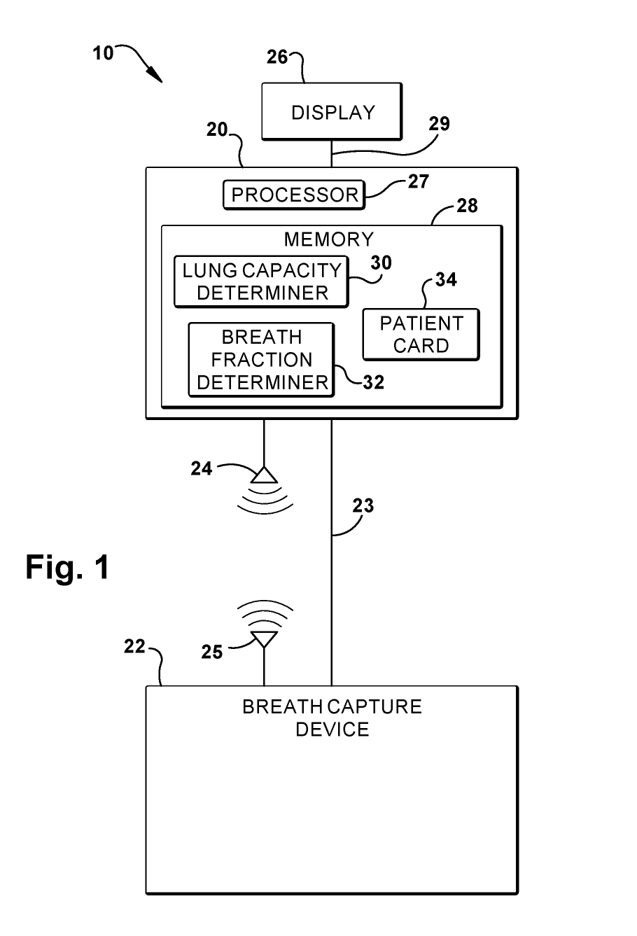

FIG. 1 provides a schematic illustration of a system 10 for use in capturing breath of a subject, in accordance with one aspect of the present invention. Breath collection system 10 includes computing device 20 in communication with breath collection device 22. Computing device 20 includes, but is not limited to a controller, a computer, a computer system etc. Computer 20 and breath collection device 22 transmit and receive messages via hard-wired connections, for example line 23, or via wireless antennas 24 and 25. The antennas can be separate or integrated with computing device 20 and breath collection device or capture device 22.

Computing device 20 includes a processor 26 and a memory 28. The memory 28 is a non-transitory, machine-readable medium that can be employed to implement systems and methods described herein, for example based on computer-executable instructions (e.g. computer logic, control logic, etc.) running on the computing device 20. The computing device 20 can be integral with the breath capture device 22 and implemented as a component of the breath capture device 22. In another example, the computing device 20 can be implemented as a stand-alone computer system and/or may operate in a networked environment and in communication with one or more general purpose networked computer systems, embedded computer systems, routers, switches, server devices, client devices, various intermediate devices/nodes. The logical connections can include a local area network (LAN) and a wide area network (WAN). In some examples, a user can enter commands and information into the computing device 20 through a user input device (not shown), such as a keyboard, a pointing device (e.g., a mouse). These and other input devices are often connected to the processor 26 through a corresponding interface that is coupled to the system. The computing device 20 is optionally connected to display 29 for review of output by computing device 20.

FIG. 1 also shows memory 28 which includes computer-executable instructions (i.e. logic) for a lung capacity determiner 30 and a breath fraction determiner 32, the details of which will be described below. Optionally, patient breath data may be recorded and accessed on a patient card 34 stored in the memory 28. The logic of the computing device 20 decodes the data in the breath characteristic signals received. The signals may be transmitted and received in an event sequence, as described further below. The computing device logic, such as that of lung capacity determiner 30 receives messages from breath collection device 22 regarding the lung capacity of a subject or patient. The computing device logic, such as that of breath capture fraction determiner 32 receives and transmits messages relating breath characteristics of the patient and determines the target sample of exhaled breath to be captured within the non-alveolar breath of the exhaled breath, for example a target sample that is within the non-alveolar fraction of exhaled breath, in another example in the first about 80% volume fraction, in another example within the first about 70% volume fraction, and in another example, within the first about 20% to about 80% volume fraction of the exhaled breath. The target sample is based at least in part on the particular target VOC, i.e. the particular breath molecule that is targeted or desired for collection or analysis, and the exhaled breath represents substantially the lung capacity of the subject. The messages or signals of the computing device 20 communicate with the breath capture device 22 to capture the target sample breath fraction of the exhaled breath.

As noted above, it is found herein that the target sample can include a target concentration of one or more target VOCs that have, for example, an elevated concentration of the one or more target VOCs. Both endogenous molecules and exogenous molecules of volatile organic compounds may exist at target concentrations, for example elevated concentrations or lowered concentrations, at different fractions of the exhaled breath. Several possible volatile organic compounds, i.e. any compound of carbon, excluding carbon monoxide, and carbon dioxide, can be targeted for analysis and/or collection, including but not limited to, nitric oxide, isoprene, beta hydroxybutyrate, 2-propanol, acetaldehyde, acetone, acetonitrile, acrylonitrile, benzene, carbon disulfide, dimethyl sulfide, ethanol, isoprene, pentane, 1-decene, 1-heptane, 1-nonene, 1-octene, 3-methylhexane, E-2-nonene, ammonia, ethane, hydrogen sulfide, triethyl amine, and trimethyl amine.

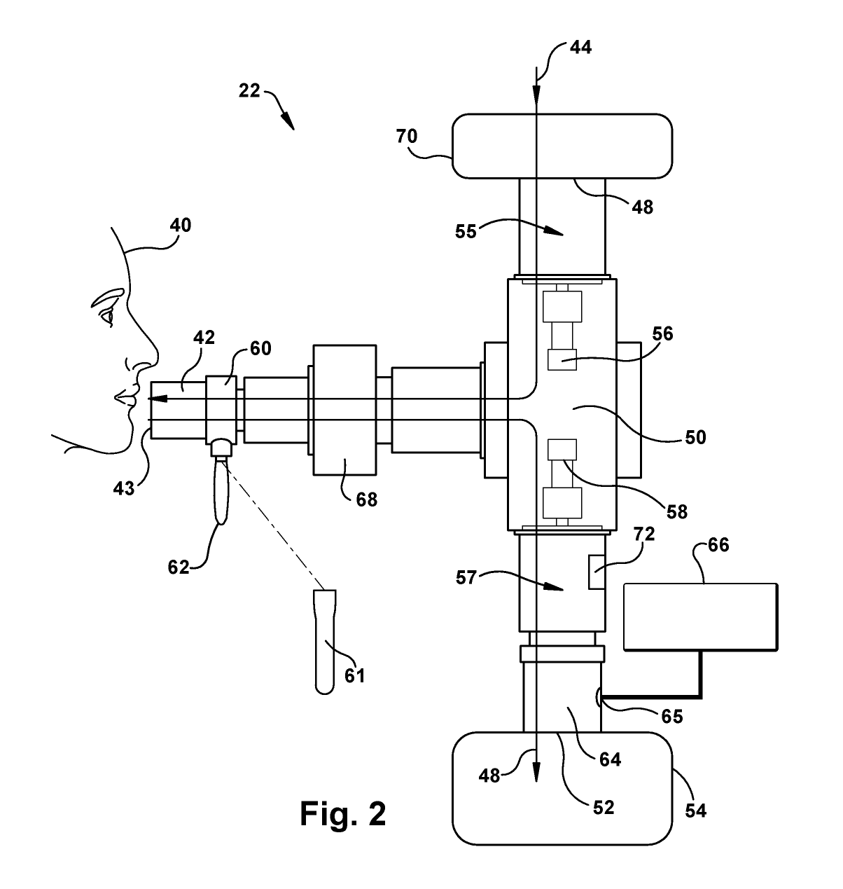

FIG. 2 illustrates a breath collection device 22 which is an example of breath collection device 22 of breath collection system 10 shown in FIG. 1. Breath collection device 22 used by patient 40 includes a mouthpiece 42 having opening 43 through which the patient can inhale air and exhale breath. Arrows 44 and 46 indicate the direction of airflow in which arrow 44 shows the path of air inhalation through inlet port 48 and into passageway 50 to mouthpiece 42, and arrow 48 shows the path of breath exhalation from mouthpiece 42 and through passageway 50 to outlet port 52 to collection chamber 54.

Pneumatically controlled valves 56 and 58 permit or prevent the passage of air flow in and out of passageway 50 which is disposed between the inlet port 48 and outlet port 52 of breath collection device 22. The pneumatically controlled valves 56, 58 are in communication with computing device 20 (FIG. 1) and open and close according to the computer-executable instructions stored on the memory 28 (FIG. 1). For example, during inhalation, valve 56 is open and valve 58 is closed to allow inhaled airflow through inlet port 48, into passageways 55 and 50, and through opening 43 of mouthpiece 42 to the mouth and lungs of subject 40. During exhalation, valve 56 is closed and valve 58 is open to allow exhaled airflow through passageway 57 and through outlet 52.

Valve 54 which can be in the open or closed position can be opened to capture breath samples for analysis. In one example, valves 56 and 58 can be programmed to open and close according to computer-executable instructions of capture fraction determiner 32 (FIG. 1). Capture fraction determiner 32 calculates the times at which valve 58 opens and closes during exhalation to capture the target sample of the exhaled breath containing a target VOC. For example, as a subject or patient exhales along flow path 48, valve 56 is closed, and valve 58 is open to allow collection of a target sample containing an target concentration of a target VOC present in the non-alveolar portion of exhaled breath, or in another example in the first about 80% volume fraction of exhaled breath. The target sample that is captured can be captured in collection chamber 54, for example. Valve 58 is closed to terminate collection of the sample at a time that is less than or equal to the time it takes for the target VOC present if the first about 80% volume fraction of exhaled breath to be collected.

In another example, breath collection device 22 includes an adaptor 64 in fluid communication with flow channels or passageways 57, and 50 when valve 58 is open. Adaptor 64 has opening 65 and which communicates with pressure transducer 66. Pressure transducer 66 receives pneumatic signals and communicates with computing device 20 to monitor pressure within flow channel 57. Breath collection device 22 optionally includes valve 60 for collecting and monitoring carbon dioxide and oxygen levels in the exhaled breath. Optional valve cap 61 is shown removed to allow diversion of a portion, for example a small portion, of exhaled breath through orifice 62. Pneumatach 68 is in fluid communication with flow channel 50 which is in communication with computing device 20 (FIG. 1) to monitor the volumetric gas flow rate of inhaled and exhaled breath through breath collection device 22. The pneumatach 68 measures the differential pressure of gas across a linear resistance. Computing device 20 can monitor and receive and transmit signals to monitor the flow parameters (e.g. volumetric flow rate) of the breath flow through the breath collection device 22. It has been found herein that consistent volumetric flow parameters of the breath given the pressure and temperature allow for accurate collection of target samples of exhaled breath containing an target concentration of the target VOC. For example, the volumetric flow rate of the exhaled breath has a flow rate that ranges from about 250 mL/sec to about 500 mL/sec, in another example from about 275 mL/sec to about 400 mL/sec, and in another example from about 250 mL/sec to about 350 mL/sec.

Breath collection device 22 optionally includes a filter 70, as shown in FIG. 2, in communication with passageway 55 and 50. The filter 70 can be used to prevent viral and bacterial exposure to the subject and to eliminate exogenous volatile organic compounds from the inhaled air. A suitable filter is N7500-2 acid gas cartridge filter, manufactured by Honeywell Corporation, for example. The exhaled breath sample can be collected into collection chamber 54 and analyzed. Collection chamber 54 can be a separate, removable compartment that is rigid or flexible, or it can be an integrated compartment of breath collection device 22. Preferably the breath sample is analyzed with an analytical device as quickly as possible, and should be analyzed within four hours.

In another aspect of the present invention, breath collection device 22 includes an analytical device 72 in communication with flow channel 57 for the measurement of gas VOCs of the exhaled breath. Examples of an analytical device include, but are not limited to, a sensor, a selected-ion flow-tube mass spectrometry (SIFT-MS), for example. Analytical device 72 can be located in a number of possible positions including, for example, inside collection chamber 54 to measure the concentration of targeted VOCs, i.e. breath molecules, in exhaled breath, or it can be disposed anywhere in the breath capture device 22 such that it is in fluid communication with exhaled breath as shown in FIG. 2. Analytical device may measure the concentration of a target VOC in a target sample remote from the breath capture device, for example a target sample that is captured in collection chamber 54 that is measured remote from the device, for example. The analytical device 72 senses the concentration of at least one target VOC in the parts per million or parts per billion. The target sample that is captured can be collected in container 54, for example, or in another example, collected in a compartment (not shown) of the breath collection device 20.

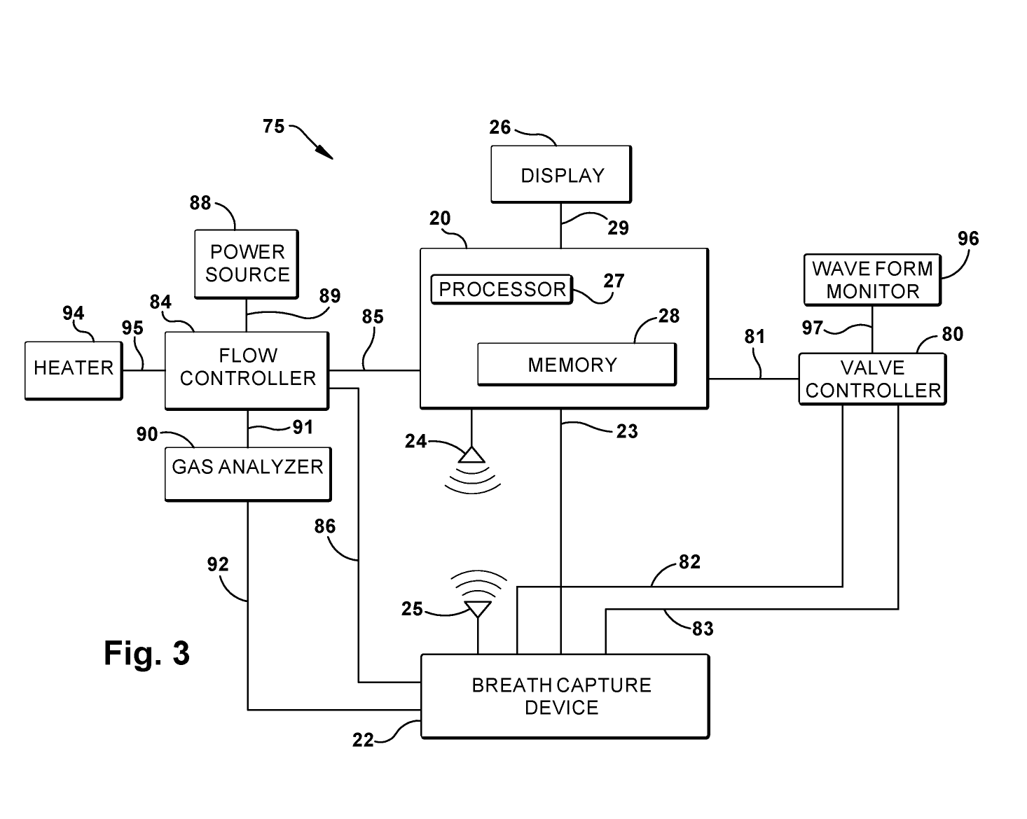

FIG. 3 is a schematic representation of a system 75 that includes a computing device 20 and a breath collection device 22, according to another example of the present invention. The computing device 20 includes processor 27 and memory 28 and operates in a networked environment using logical connections to one or more computers or systems. For example, FIG. 3 shows computing device 20 is connected to display 26 via logical connection 29, connected to breath capture device 22 via logical connection 23, connected to valve controller 80 via logical connection 81, and connected to the flow controller 84 via logical connection 85. Computing device 20 can also communicate with breath capture device 22 wirelessly via antennas 24 and 25. The computing device 20 can be a computer or controller, for example, and is configured to determine a capture fraction of an exhaled breath. The capture fraction is a target sample containing a target concentration of a target VOC within the non-alveolar portion of the exhaled breath, for example the first about 80% volume fraction of the exhaled breath of a subject where the exhaled breath represents substantially the lung capacity of the subject. The control logic in the memory 28 of computing device 20 decodes data from the breath capture device directly or via other systems. Optionally, a remote computer can be connected to computing device 20, for example, a workstation, a computer system, a router, a peer device or other common network node, and typically includes many or all of the elements described relative to system 75. The logical connections can include a local area network (LAN) and a wide area network (WAN).

Flow controller 84 powered by power source 88 is connected to heater 94 via logical connection 95, and is connected to gas analyzer 90 via logical connection 91. Pneumatic valve controller 80 is connected to wave form monitor 96 via logical connection 97. Flow controller 84 is connected to breath capture device 22 via logical connection 86 and monitors the flow characteristics of breath flowing through the breath capture device 22.

Valve controller 80 is connected to pneumatic valves 56 and 58 (FIG. 2) and can allow or prevent the passage of air flow in and out of passageway 50 at various breath fractions of exhaled breath to collect target samples as described above.

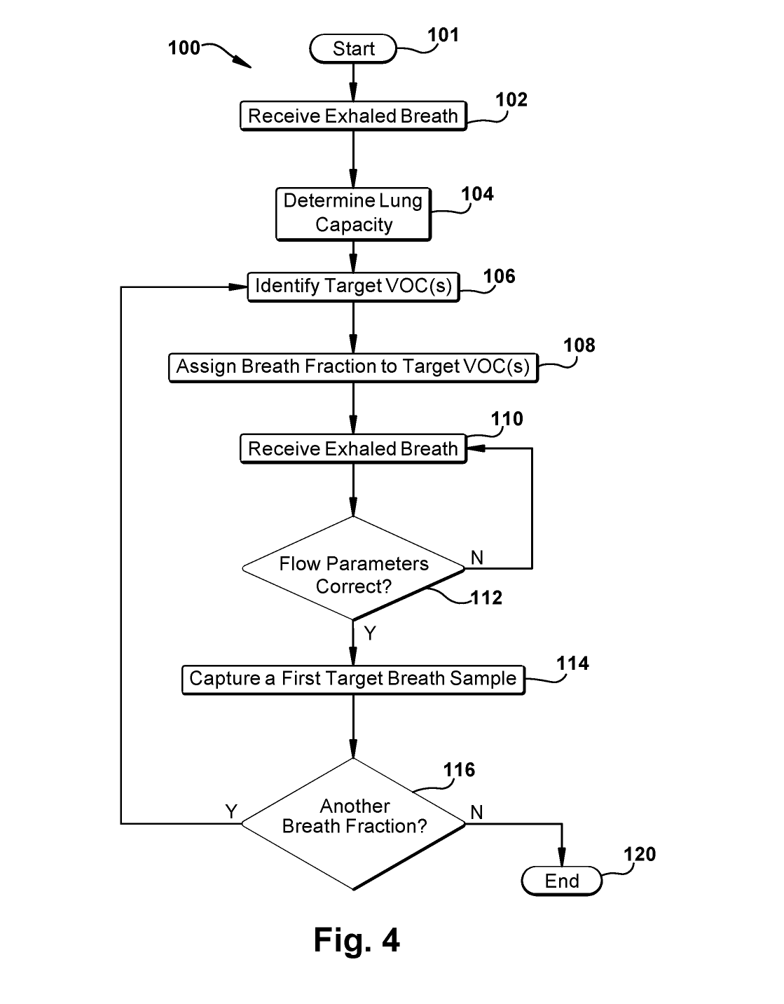

FIG. 4 is a flow chart illustrating a method 100 of implemented by computing device 20 of FIGS. 1 and 3, according to examples of the present invention. As mentioned above, the computing device 20 can monitor inhaled and exhaled breath parameters of a subject and receives data for each subject in a usable format to which breath collection system 10 is configured. FIG. 4 illustrates an example method 100 by which a computing device 20 executes instructions via non-transitory machine-readable medium from the signals received by the breath collection device 22. At the start depicted at box 101, signals relating to the inhaled and exhaled breath are received at box 102 when the subject or patient 40 (FIG. 2) inhales and exhales air through breath collection device 22. At box 104 lung capacity determiner 30 (FIG. 2) determines the lung capacity or forced vital capacity of the subject as described above. For example, lung capacity determiner 30 (FIG. 1) calculates the volume of air exhaled after the subject inhales all the air that can be inhaled naturally. This can be calculated in a variety of ways, such as for example, by measuring the volumetric flow rate of the air exhaled and the volumetric flow rate is then multiplied by the time for exhalation. In another example, the lung capacity determiner can calculate the volume of air inhaled by measuring the volumetric flow rate of inhaled air and multiplying by the time of inhalation.

With reference to FIG. 4, the lung capacity of the subject is determined at 104, and the breath fraction determiner 32 (FIG. 1) determines the volume fraction of the exhaled breath or the target sample to be captured. The target sample having an target concentration of the target VOC is captured in the non-alveolar portion of the exhaled breath, and in another example the first 80% volume fraction of the exhaled breath. Exhaled breath can include a myriad of several volatile organic compounds, i.e. any compound of carbon, excluding carbon monoxide, and carbon dioxide. Examples of target volatile organic compounds include, but are not limited to, 2-propanol, acetaldehyde, acetone, acetonitrile, acrylonitrile, benzene, carbon disulfide, dimethyl sulfide, ethanol, isoprene, pentane, 1-decene, 1-heptane, 1-nonene, 1-octene, 3-methylhexane, E-2-nonene, ammonia, ethane, hydrogen sulfide, triethyl amine, and trimethyl amine.

In another example, the volume fraction of exhaled breath from which the target sample is to be captured can be a smaller subset volume fraction of the non-alveolar fraction of the exhaled breath or a subset fraction of the first about 80% volume fraction of exhaled breath. In such case, the target sample containing an target concentration of the target VOC can be determined, at least in part, by the identity of the target VOC to be analyzed or collected. Capture fracture determiner 32 receives an input identifying the target VOC as depicted at box 106. For example, if acetone is the target VOC then the capture fraction determiner 32 will receive input that the preselected compound is acetone. The capture fraction determiner 32 can include a look-up table that lists several target VOCs and the respective the volume fraction of exhaled breath that contains target concentrations for each target VOC, from which the target sample is captured within the non-alveolar fraction of exhaled breath, and in another example the first about 80% volume fraction of exhaled breath. For example, if the preselected or target VOC is acetone the capture fraction determiner 32 can receive input from the look-up table that the target concentration of acetone can be captured in the first about 60% volume fraction of exhaled breath, and in another example, in the first 40% to about 60% volume fraction of exhaled breath.

The breath fraction determiner 32 also receives the lung capacity determined by lung capacity determiner 30 as described above. Utilizing information from breath fraction determiner 32, for example, the identity of the target VOC and the lung capacity of the subject, the breath fraction determiner 32 determines and/or assigns the volume fraction to be captured. In other words, the breath fraction determiner 32 performs logical functions to determine the portion of the exhaled breath fraction to capture for a given target VOC and which is, for example in the non-alveolar fraction of breath, in another example in the first about 80% volume fraction of exhaled breath, or in another example a volume fraction that is a subset of these fractions of exhaled breath. From at least this collective information the breath fraction determiner 32 determines the interval of time, for example the starting time and duration at which valve 58 (FIG. 2) is opened while valve 56 is closed, to collect the target sample of the target VOC as depicted at box 108.

As mentioned above, a target sample can include a volume fraction of exhaled breath that is a subset of the non-alveolar portion of exhaled breath, or a subset of the first about 80% volume fraction of exhaled breath can be collected. The data in the look-up table indicating the target VOCs and the corresponding volume fraction of exhaled breath to be analyzed and/or collected can be based on findings from study of exhaled breath of several volatile organic compounds, for example those provided in the Examples below. Target samples that are subset fractions of the first about 80% volume fraction of the exhaled breath, include target samples collected in the first about 70% volume fraction of exhaled breath, in another example, in the first about 60% volume fraction, in another example in the first about 40% volume fraction, and in another example, in the about first about 20% volume fraction of exhaled breath. Additional subset fractions of exhaled breath include, but are not limited to, the first about 20% to about the 80% volume fraction, in another example, the first about 20% to about 60% volume fraction, in another example, the first about 20% to about 40% volume fraction, in another example the first about 40% to about 80% volume fraction, in another example, the first about 40% to about 60% volume fraction, and in another example, the first about 60% to about 80% volume fraction of exhaled breath.

Target VOCs in the above-listed subset fractions can include any compound of carbon, excluding carbon monoxide, and carbon dioxide, including but not limited to, nitric oxide, isoprene, beta hydroxybutyrate, 2-propanol, acetaldehyde, acetone, acetonitrile, acrylonitrile, benzene, carbon disulfide, dimethyl sulfide, ethanol, isoprene, pentane, 1-decene, 1-heptane, 1-nonene, 1-octene, 3-methylhexane, E-2-nonene, ammonia, ethane, hydrogen sulfide, triethyl amine, and trimethyl amine, for example. Target VOCs having target concentrations when captured in the first about 60% volume fraction of exhaled breath can include, but are not limited to, 2-propanol, acetaldehyde, acetone, acetonitrile, acrylonitrile, benzene, carbon disulfide, dimethyl sulfide, ethanol, isoprene, pentane, 3-methylhexane, E-2-nonene, ethane, hydrogen sulfide, triethyl amine, and trimethyl amine, for example. Target VOCs having target concentrations when captured in the first about 40% volume fraction of exhaled breath can include, but are not limited to, acetone, carbon disulfide, ethanol, E-2-nonene, ethane, and triethyl amine, for example. Target VOCs having a target concentration, for example an elevated concentration, when captured in the first about 20% volume fraction of exhaled breath can include, but are not limited to, acetone, ethane, and triethyl amine, for example.

Target VOCs having a target concentration, for example an elevated concentration when captured in the first about 20% to about the first about 80% volume fraction of exhaled breath can include, but are not limited to, 2-propanol, acetaldehyde, acetone, acetonitrile, acrylonitrile, benzene, carbon disulfide, dimethyl sulfide, ethanol, isoprene, pentane, 1-decene, 1-heptane, 1-nonene, 1-octene, 3-methylhexane, E-2-nonene, ammonia, ethane, hydrogen sulfide, triethyl amine, and trimethyl amine, for example. Target VOCs having a target concentration, for example an elevated concentration when captured in the first about 20% to the first about 60% volume fraction of exhaled breath can include, but are not limited to, 2-propanol, acetaldehyde, acetone, acetonitrile, acrylonitrile, benzene, carbon disulfide, dimethyl sulfide, ethanol, isoprene, pentane, 3-methylhexane, E-2-nonene, ethane, hydrogen sulfide, triethyl amine, and trimethyl amine, for example. Target VOCs having a target concentration, for example an elevated concentration when captured in the first about 20% to the first about 40% volume fraction of exhaled breath can include, but are not limited to, acetone, carbon disulfide, ethanol, E-2-nonene, ethane, and triethyl amine, for example. Target VOCs having a target concentration, for example an elevated concentration when captured in the first about 40% to the first about 80% volume fraction of exhaled breath can include, but are not limited to, 2-propanol, acetaldehyde, acetone, acetonitrile, acrylonitrile, benzene, carbon disulfide, dimethyl sulfide, ethanol, isoprene, pentane, 1-decene, 1-heptane, 1-nonene, 1-octene, 3-methylhexane, E-2-nonene, ammonia, ethane, hydrogen sulfide, triethyl amine, and trimethyl amine, for example. Target VOCs having a target concentration, for example an elevated concentration when captured in the first about 40% to the first about 60% volume fraction of exhaled breath can include, but are not limited to, 2-propanol, acetaldehyde, acetone, acetonitrile, acrylonitrile, benzene, carbon disulfide, dimethyl sulfide, ethanol, isoprene, pentane, 3-methylhexane, E-2-nonene, ethane, hydrogen sulfide, and trimethyl amine, for example. In another example, target VOCs having a target concentration, for example an elevated concentration when captured in the first about 60% to the first about 80% volume fraction of exhaled breath can include, but are not limited to, 2-propanol, acetaldehyde, acetone, acetonitrile, acrylonitrile, benzene, carbon disulfide, dimethyl sulfide, ethanol, isoprene, pentane, 1-decene, 1-heptane, 1-nonene, 1-octene, 3-methylhexane, E-2-nonene, ammonia, ethane, hydrogen sulfide, triethyl amine, and trimethyl amine, for example.

Multiple target VOCs can be captured and concentrations measured in any of the volume breath fractions and subset fractions of non-alveolar breath of the exhaled breath fractions described above. For example, a single target sample captured within the first about 80% volume fraction of exhaled breath can include a target concentration, for example an elevated concentration of more than one target VOC. Likewise, for example, a single target sample captured within the non-alveolar exhaled breath, or in the first about 80% volume fraction of exhaled breath, for example, can include an lowered concentration of more than one target VOC.

In another example, two or more target samples are collected and the target sample comprises a second target concentration of a second target VOC where the second target VOC is a different compound than the first target VOC.

In other words, multiple target samples of different target VOCs may be collected in a single exhaled breath or two separate exhaled breaths. The two or more samples can be captured from the first about 80% volume fraction of the exhaled breath, in another example, in the first about 70% volume fraction of exhaled breath, in another example, in the first about 60% volume fraction of exhaled breath, in another example, in the first about 20% to the first about 80% volume fraction of exhaled breath, in another example, in the first about 20% to the first about 60% volume fraction, in another example in the first about 40% to the first about 60% volume fraction, and in another example, the about 40% to about 80% volume fraction. A second target VOC can be from a different fraction than the first target VOC of the exhaled breath. The size of the target sample can vary and can represent, for example, up to about 10% by volume of the exhaled breath, in another example up to about 20% by volume of the exhaled breath, in another example up to about 35% by volume of exhaled breath, in another example from about 5% to about 30% by volume of the exhaled breath, in another example from about 10% to about 25% by volume of exhaled breath, in another example from about 15% to about 25% by volume of exhaled breath, and in yet another example from about 18% to about 22% by volume of exhaled breath.

Box 110 of FIG. 4 indicates the step of receiving or capturing the exhaled breath, for example collection of the target VOC in collection chamber 54. The computing device depicted at box 112 determines whether the flow parameters of the exhaled breath are within defined parameters described herein. In any of the example methods described above, the computing device can receive data relating to pressure, temperature and volumetric flow rate. As an example, the volumetric flow rate data of exhaled breath of the subject can range from about 250 mL/second to about 500 mL/second and is within the defined flow parameters. In another example, the volumetric flow rates ranges from about 300 mL/sec to about 450 mL/sec, in another example from about 325 mL/sec to about 400 mL/sec, and in another example from about 250 mL/sec to about 350 mL/sec. At step 112 a query is made as to whether the exhaled breath is within the specified or predetermined volumetric flow rate range. If the data generated regarding the exhaled breath is outside the flow parameters then the computing device can signal as such and look to receive data from another exhaled breath back at step 110. If the flow parameters are within range then at step 114 the breath fraction determiner 32 determines the fraction(s) of exhaled breath to capture and sends a signal, for example, to the breath collection device 22 (FIG. 1), a controller, valves, etc., so that the target sample(s) containing the target concentration of the VOC(s) may be captured.

Next, at 116 a query is made as to whether another (e.g. a second) target sample having a different target VOC of the exhaled breath is targeted for collection. If a second target VOC that is different than the first target VOC is targeted for collection then the breath fraction determiner will identify the target VOC at step 108 and the method will be repeated from step 108 as described above. When there are no more target VOCs and breath fractions to be captured then the method ends at 120. Accordingly, in one example two or more target VOCs may be collected in the same target sample of exhaled breath or two or more target VOCs may be collected at two or more different fractions of the exhaled breath. If one or more additional target samples of additional target VOC(s) are targeted for collection, the breath capture determiner 32 will determine the next volume fraction and a second target breath sample will be collected by breath capture device at step 114. As an example, the first target sample can be the first about 20% to about 60% of the exhaled breath having a target concentration, for example an elevated concentration, of a first target VOC, for example acetone, and a second target sample can be of the first about 60% to about 80% of the exhaled breath having a second target concentration, for example an elevated concentration, of a second target VOC, for example 1-Decene. Breath capture determiner 32 will iteratively determine whether additional target samples within the first about 80% volume fraction of exhaled breath is needed for collection. Signals will be sent from the breath capture determiner 32 to valve(s), e.g. valve 58, to be opened and closed at the appropriate times to collect and terminate collection, respectively, the target sample within the first about 80% volume fraction, or any appropriate subset thereof, of the exhaled breath. In another example, a first target sample having a first target VOC can be collected in a first exhaled breath and a second target sample of having a second target VOC can be collected in a second exhaled breath of the subject.

Examples have been included to more clearly describe several example embodiments of the present invention and associated methods and/or operational advantages. However, there are a wide variety of other example embodiments within the scope of the present invention, which should not be limited to the particular examples provided herein.

### EXAMPLES

The following examples were conducted to determine the concentrations of various target breath compounds using an equipment set-up with breath collection device 22 shown in FIG. 2 and system 75 shown in FIG. 3.

Nine non-smoking healthy individuals were recruited (five male, mean age 27 yrs). The study was approved by the Cleveland Clinic IRB. Prior to gas analysis each individual completed spirometry testing to identify forced vital capacity (VC) or lung capacity from total lung capacity (TLC) to residual volume (RV). The lung capacity or forced vital capacity was divided into five portions. Then each individual inhaled through a filter from RV to TLC, to remove ambient VOC's and then exhaled at a flow rate of 350 ml/sec. The five (5) fractions of the exhaled breath are identified in Tables 2 to 24 below as fractions a, b, c, d, and e. Fraction “a” represents the first one fifth (i.e. greater than about 0% to about 20%) of the exhaled breath collected, fraction “b” represents the second one fifth (i.e. greater than about 20% to about 40%) of the exhaled breath collected, fraction “c” represents the third one fifth (i.e. greater than about 40% to about 60%) of the exhaled breath collected, fraction “d” represents the fourth one fifth (i.e. greater than about 60% to about 80%) of the exhaled breath collected, and fraction “e” represents the last one fifth (i.e. greater than about 80% to about 100%) of the exhalation. Data were recorded using a program written in LabView® (National Instruments). Fractionation was accomplished by using a Hans Rudolph® “manual valve switch” programmed to adjust for lung volumes. Each individual also had a Full mixed (offline) exhalation and an online sample exhaled directly into the Syft® mass spectrometer. Oxygen, carbon dioxide, heart rate, respiratory rate, oxygen saturation, flow and tidal volumes were also monitored during testing.

Lung volume “a”, which contains anatomical dead space had relatively lower concentration of VOCs. Surprisingly, lung volume “e” which represented end expiration gas, and by some standards characterized as alveolar gas, was also lower. Highest concentrations for several volatile organic compounds where identified in fraction “d” that represents the area of the fourth portion or fraction of lung capacity in the exhaled breath. A full breath offline had better correlation than online to fraction “d”.

In the following Examples 1-22 several volatile organic breath compounds or “target molecules” or “target VOCs” were analyzed by capturing several fractions of the exhaled breath stream of the same target molecule and quantifying the amount of the molecule in the different fractions of the breath. The target volatile organic compounds captured were as follows: 2-propanol, acetaldehyde, acetone, acetonitrile, acrylonitrile, benzene, carbon disulfide, dimethyl sulfide, ethanol, isoprene, pentane, 1-decene, 1-heptane, 1-nonene, 1-octene, 3-methylhexane, E-2-nonene, ammonia, ethane, hydrogen sulfide, triethyl amine, and trimethyl amine. The results of the quantified values based on the greatest median number of molecules, the greatest maximum number of molecules, or “peak” number of molecules, and the greatest mean number of molecules, for each of the five breath fractions of the above compounds are listed in Tables 1-22. The results of full-breath and on-line tests are also listed.

Selected-ion flow-tube mass spectrometry (SIFT-MS) provides precise identification of trace gases in the human breath in the parts per trillion ranges. Using selected-ion flow-tube mass spectrometry, precise identification of VOC in the breath in the parts per billion range (ppb) was achieved on all subjects.

A summary of the results of the volume fractions of exhaled breath that contained the target concentrations of target VOCs are listed in Table 23 and show that various VOCs had the highest concentration at specific fractions of the exhaled breath, and that these fractions were within the first 80% breath stream fraction of the exhaled breath.

### Example 1

The results of breath analysis for breath compound 2-propanol are shown in Table 1. The greatest median number of molecules was found in the first >60%-80% fraction of exhaled breath, the maximum number of molecules was found in the first >40%-60% fraction of the exhaled breath, and the greatest mean number of molecules was found in the first >60%-80% fraction of exhaled breath.

TABLE 1

| | | | | | Std. |
|---|---|---|---|---|---|
| Fraction | Minimum | Median | Maximum | Mean | Dev. |
| a | 16.8 | 38.34 | 62.03 | 35.6000 | 15.0883 |
| (>0-20%) | | | | | |
| b | 25.88 | 48.28 | 66.74 | 49.6489 | 15.2303 |
| (>20-40%) | | | | | |
| c | 31.71 | 48.77 | 99.59 | 51.4200 | 20.0298 |
| (>40-60%) | | | | | |
| d | 21.82 | 53.25 | 89.61 | 52.5067 | 19.0832 |
| (>60-80%) | | | | | |
| e | 24.99 | 38.57 | 53.81 | 39.5922 | 8.9159 |
| (>80-100%) | | | | | |
| Full Breath | 14.17 | 28.87 | 80.2 | 35.0956 | 23.0439 |
| Online | 5.14 | 6.2 | 16.3 | 7.8422 | 3.5752 |

### Example 2

The results of breath analysis for breath compound acetaldehyde are shown in Table 2. The greatest median number of molecules was found in the first >60%-80% fraction of exhaled breath, the maximum number of molecules was found in the first >40%-60% fraction of the exhaled breath, and the greatest mean number of molecules was found in the first >40%-60% fraction of exhaled breath.

TABLE 2

| | | | | | Std. |
|---|---|---|---|---|---|
| Fraction | Min. | Median | Max. | Mean | Dev. |
| a | 10.79 | 18.15 | 32.63 | 19.0756 | 8.1276 |
| (>0-20%) | | | | | |
| b | 13.7 | 26.78 | 49.56 | 26.8256 | 10.3283 |
| (>20-40%) | | | | | |
| c | 18.51 | 28.24 | 66.85 | 32.5400 | 14.6921 |
| (>40-60%) | | | | | |
| d | 11.58 | 30.79 | 55.36 | 30.7967 | 13.0828 |
| (>60-80%) | | | | | |
| e | 9.09 | 17.73 | 37.24 | 20.4189 | 8.3001 |
| (>80-100%) | | | | | |
| Full Breath | 9.31 | 12.23 | 53.5 | 19.2967 | 15.3952 |
| Online | 11.8 | 14.8 | 33.3 | 17.1333 | 6.5985 |

### Example 3

The results of breath analysis for the breath compound acetone are shown in Table 3. The greatest median number of molecules was found in the first >60%-80% fraction of exhaled breath, the maximum number of molecules was found in the first >0%-20% fraction of the exhaled breath, and the greatest mean number of molecules was found in the first >40%-60% fraction of exhaled breath.

TABLE 3

| Fraction | Minimum | Median | Maximum | Mean | Std. Dev. |
|---|---|---|---|---|---|
| a | 5.53 | 24.38 | 223.52 | 55.696 | 69.807 |
| (>0-20%) | | | | | |
| b | 23.54 | 31.68 | 81.47 | 43.187 | 21.685 |
| (>20-40%) | | | | | |
| c | 20.92 | 55.3 | 177.68 | 74.056 | 56.676 |
| (>40-60%) | | | | | |
| d | 8.27 | 47.53 | 119.31 | 61.671 | 44.569 |
| (>60-80%) | | | | | |
| e | 4.4 | 26.9 | 188.23 | 47.042 | 56.074 |
| (>80-100%) | | | | | |
| Full Breath | 17.46 | 44.72 | 545.98 | 107.213 | 167.936 |
| Online | 88.2 | 187 | 676 | 228.356 | 174.315 |

### Example 4

The results of breath analysis for breath compound acetonitrile are shown in Table 4. The greatest median number of molecules was found in the first >40%-60% fraction of exhaled breath, the maximum number of molecules was found in the first >60%-80% fraction of the exhaled breath, and the greatest mean number of molecules was found in the first >60%-80% fraction of exhaled breath.

TABLE 4

| Fraction | Minimum | Median | Maximum | Mean | Std. Dev. |
|---|---|---|---|---|---|
| a | 5.42 | 8.63 | 10.84 | 8.1744 | 2.0768 |
| (>0-20%) | | | | | |
| b | 5.7 | 14.28 | 24.58 | 13.3089 | 5.7240 |
| (>20-40%) | | | | | |
| c | 6.2 | 16.74 | 30.04 | 15.5900 | 7.7804 |
| (>40-60%) | | | | | |
| d | 4.62 | 12.49 | 50.57 | 18.3311 | 14.1505 |
| (>60-80%) | | | | | |
| e | 2.81 | 9.81 | 13.69 | 8.8789 | 4.0019 |
| (>80-100%) | | | | | |
| Full Breath | 3.63 | 5.09 | 28.93 | 9.3222 | 8.4315 |
| Online | 0.42 | 0.52 | 0.64 | 0.5211 | 0.0677 |

### Example 5

The results of breath analysis for breath compound acrylonotrile are shown in Table 5. The greatest median number of molecules was found in the first >60%-80% fraction of exhaled breath, the maximum number of molecules was found in the first >40%-60% fraction of the exhaled breath, and the greatest mean number of molecules was found in the first >60%-80% fraction of exhaled breath.

TABLE 5

| Fraction | Minimum | Median | Maximum | Mean | Std. Dev. |
|---|---|---|---|---|---|
| a | 0.45 | 0.62 | 0.79 | 0.617778 | 0.104496 |
| (>0-20%) | | | | | |
| b | 0.46 | 0.71 | 0.97 | 0.721111 | 0.147770 |
| (>20-40%) | | | | | |
| c | 0.43 | 0.67 | 1.03 | 0.723333 | 0.194808 |
| (>40-60%) | | | | | |
| d | 0.56 | 0.77 | 0.87 | 0.731111 | 0.125044 |
| (>60-80%) | | | | | |
| e | 0.49 | 0.57 | 0.88 | 0.620000 | 0.138744 |
| (>80-100%) | | | | | |
| Full Breath | 0.31 | 0.54 | 0.74 | 0.510000 | 0.135370 |
| Online | — | — | — | — | — |

### Example 6

The results of breath analysis for breath compound benzene are shown in Table 6. The greatest median number of molecules was found in the first >40%-60% fraction of exhaled breath, the maximum number of molecules was found in the first >40%-60% fraction of the exhaled breath, and the greatest mean number of molecules was found in the first >40%-60% fraction of exhaled breath.

TABLE 6

| Fraction | Minimum | Median | Maximum | Mean | Std. Dev. |
|---|---|---|---|---|---|
| a | 1.13 | 1.45 | 2.24 | 1.58778 | 0.402144 |
| (>0-20%) | | | | | |
| b | 1.21 | 1.55 | 2.11 | 1.57778 | 0.332445 |
| (>20-40%) | | | | | |
| c | 1.18 | 1.68 | 2.61 | 1.70111 | 0.441147 |
| (>40-60%) | | | | | |
| d | 0.72 | 1.58 | 1.99 | 1.41111 | 0.373846 |
| (>60-80%) | | | | | |
| e | 1 | 1.47 | 2.09 | 1.48333 | 0.348102 |
| (>80-100%) | | | | | |
| Full Breath | 0.84 | 1.35 | 2.11 | 1.47333 | 0.418868 |
| Online | 0.71 | 0.88 | 1.53 | 0.93333 | 0.257488 |

### Example 7

The results of breath analysis for breath compound carbon disulfide are shown in Table 7. The greatest median number of molecules was found in the first >20%-40% fraction of exhaled breath, the maximum number of molecules was found in the first >40%-60% fraction of the exhaled breath, and the greatest mean number of molecules was found in the first >40%-60% fraction of exhaled breath.

TABLE 7

| Fraction | Minimum | Median | Maximum | Mean | Std. Dev. |
|---|---|---|---|---|---|
| a | 0.87 | 1.09 | 1.69 | 1.24222 | 0.311800 |
| (>0-20%) | | | | | |
| b | 0.64 | 1.43 | 2.09 | 1.37000 | 0.415812 |
| (>20-40%) | | | | | |
| c | 0.75 | 1.15 | 3.45 | 1.45778 | 0.878006 |
| (>40-60%) | | | | | |
| d | 0.81 | 1.27 | 1.92 | 1.35000 | 0.408901 |
| (>60-80%) | | | | | |
| e | 0.44 | 1.3 | 1.71 | 1.21778 | 0.457050 |
| (>80-100%) | | | | | |
| Full Breath | 0.82 | 1.41 | 2.11 | 1.45778 | 0.486846 |
| Online | 0.67 | 0.82 | 0.94 | 0.80111 | 0.096105 |

### Example 8

The results of breath analysis for breath compound dimethyl sulfide are shown in Table 8. The greatest median number of molecules was found in the first >80%-100% fraction of exhaled breath, the maximum number of molecules was found in the first >60%-80% fraction of the exhaled breath, and the greatest mean number of molecules was found in the first >40%-60% fraction of exhaled breath.

TABLE 8

| Fraction | Minimum | Median | Maximum | Mean | Std. Dev. |
|---|---|---|---|---|---|
| a | 0.32 | 0.55 | 0.88 | 0.54222 | 0.15810 |
| (>0-20%) | | | | | |
| b | 0.42 | 0.59 | 1.61 | 0.68778 | 0.35999 |
| (>20-40%) | | | | | |
| c | 0.44 | 0.6 | 1.51 | 0.75222 | 0.36952 |
| (>40-60%) | | | | | |
| d | 0.23 | 0.58 | 1.52 | 0.70333 | 0.37047 |
| (>60-80%) | | | | | |
| e | 0.18 | 0.63 | 0.98 | 0.60556 | 0.27162 |
| (>80-100%) | | | | | |
| Full Breath | 0.44 | 0.62 | 2.07 | 0.86444 | 0.52381 |
| Online | 0.35 | 1.05 | 4.33 | 1.49667 | 1.30512 |

### Example 9

The results of breath analysis for breath compound ethanol are shown in Table 9. The greatest median number of molecules was found in the first >40%-60% fraction of exhaled breath, the maximum number of molecules was found in the first >20%-40% fraction of the exhaled breath, and the greatest mean number of molecules was found in the first >20%-40% fraction of exhaled breath.

TABLE 9

| | | | | | Std. |
|---|---|---|---|---|---|
| Fraction | Min. | Median | Max. | Mean | Dev. |
| a | 20.37 | 25.92 | 223.6 | 50.8156 | 65.5132 |
| (>0-20%) | | | | | |
| b | 21.34 | 36.77 | 253.08 | 61.2700 | 73.4326 |
| (>20-40%) | | | | | |
| c | 23.18 | 44.33 | 114.37 | 53.1211 | 31.0927 |
| (>40-60%) | | | | | |
| d | 25.94 | 32.59 | 64.9 | 36.2556 | 12.2223 |
| (>60-80%) | | | | | |
| e | 22.3 | 32.55 | 140.33 | 49.2200 | 36.8654 |
| (>80-100%) | | | | | |
| Full Breath | 21.05 | 62.98 | 95.73 | 56.9900 | 28.2054 |
| Online | 10.7 | 28.8 | 245 | 53.1778 | 74.6223 |

### Example 10

The results of breath analysis for breath compound isoprene are shown in Table 10. The greatest median number of molecules was found in the first >40%-60% fraction of exhaled breath, the maximum number of molecules was found in the first >60%-80% fraction of the exhaled breath, and the greatest mean number of molecules was found in the first >40%-60% fraction of exhaled breath.

TABLE 10

| | | | | | Std. |
|---|---|---|---|---|---|
| Fraction | Min. | Median | Max. | Mean | Dev. |
| a | 1.57 | 3.84 | 8.5 | 4.6189 | 2.51960 |
| (>0-20%) | | | | | |
| b | 2.37 | 3.64 | 9.08 | 4.7067 | 2.54497 |
| (>20-40%) | | | | | |
| c | 3.35 | 5.16 | 8.27 | 5.4033 | 1.85536 |
| (>40-60%) | | | | | |
| d | 2.36 | 3.55 | 9.96 | 4.9478 | 2.81969 |
| (>60-80%) | | | | | |
| e | 1.48 | 4.18 | 6.67 | 3.9233 | 1.90993 |
| (>80-100%) | | | | | |
| Full Breath | 1.62 | 4.74 | 8.28 | 4.9778 | 2.20291 |
| Online | 16 | 22.6 | 28.6 | 22.7000 | 3.89744 |

### Example 11

The results of breath analysis for breath compound pentane are shown in Table 11. The greatest median number of molecules was found in the first >60%-80% fraction of exhaled breath, the maximum number of molecules was found in the first >40%-60% fraction of the exhaled breath, and the greatest mean number of molecules was found in the first >60%-80% fraction of exhaled breath.

TABLE 11

| | | | | | Std. |
|---|---|---|---|---|---|
| Fraction | Min. | Median | Max. | Mean | Dev. |
| a | 3.96 | 7.33 | 13.33 | 7.4644 | 2.95122 |
| (>0-20%) | | | | | |
| b | 5.52 | 9.84 | 14.41 | 9.7656 | 2.84631 |
| (>20-40%) | | | | | |
| c | 6.48 | 10.01 | 25.67 | 11.7244 | 6.21007 |
| (>40-60%) | | | | | |
| d | 3.26 | 10.54 | 24.88 | 11.7389 | 6.27014 |
| (>60-80%) | | | | | |
| e | 3.82 | 7.1 | 15.3 | 7.5878 | 3.25471 |
| (>80-100%) | | | | | |
| Full Breath | 4.69 | 6.24 | 25.41 | 9.5889 | 6.83926 |
| Online | 10.9 | 15.4 | 35.9 | 16.7222 | 7.80861 |

### Example 12

The results of breath analysis for breath compound 1-decene are shown in Table 12. The greatest median number of molecules was found in the first >60%-80% fraction of exhaled breath, the maximum number of molecules was found in the first >60%-80% fraction of the exhaled breath, and the greatest mean number of molecules was found in the first >60%-80% fraction of exhaled breath.

TABLE 12

| | | | | | Std. |
|---|---|---|---|---|---|
| Fraction | Min. | Median | Max. | Mean | Dev. |
| a | 4.52 | 5.91 | 12.29 | 7.2233 | 3.0645 |
| (>0-20%) | | | | | |
| b | 3.6 | 12.3 | 31.5 | 13.1289 | 8.5743 |
| (>20-40%) | | | | | |
| c | 9.22 | 14.07 | 29.56 | 17.5667 | 8.4874 |
| (>40-60%) | | | | | |
| d | 3.4 | 16.34 | 59.31 | 21.6144 | 18.4050 |
| (>60-80%) | | | | | |
| e | 4.2 | 10.79 | 22.22 | 11.0444 | 5.8703 |
| (>80-100%) | | | | | |
| Full Breath | 3.6 | 4.48 | 32.36 | 10.2611 | 10.0884 |
| Online | 1.55 | 3.5 | 4.32 | 3.3967 | 0.8572 |

### Example 13

The results of breath analysis for breath compound 1-heptene are shown in Table 13. The greatest median number of molecules was found in the first >60%-80% fraction of exhaled breath, the maximum number of molecules was found in the first >60%-80% fraction of the exhaled breath, and the greatest mean number of molecules was found in the first >60%-80% fraction of exhaled breath.

TABLE 13

| | | | | | Std. |
|---|---|---|---|---|---|
| Fraction | Min. | Median | Max. | Mean | Dev. |
| a | 5.17 | 7.23 | 8.52 | 7.06111 | 1.11223 |
| (>0-20%) | | | | | |
| b | 4.8 | 8.65 | 11.01 | 8.63889 | 1.97452 |
| (>20-40%) | | | | | |
| c | 4.69 | 8.93 | 13.9 | 9.51222 | 2.86506 |
| (>40-60%) | | | | | |
| d | 5.01 | 7.96 | 13.49 | 8.60111 | 2.79833 |
| (>60-80%) | | | | | |
| e | 4.02 | 7.13 | 8.04 | 6.71333 | 1.37893 |
| (>80-100%) | | | | | |
| Full Breath | 4.43 | 6.33 | 10.12 | 6.99444 | 2.07171 |
| Online | 4.08 | 5.5 | 12.1 | 6.60333 | 2.65384 |

### Example 14

The results of breath analysis for breath compound 1-nonene are shown in Table 14. The greatest median number of molecules was found in the first >60%-80% fraction of exhaled breath, the maximum number of molecules was found in the first >60%-80% fraction of the exhaled breath, and the greatest mean number of molecules was found in the first >60%-80% fraction of exhaled breath.

TABLE 14

| | | | | | Std. |
|---|---|---|---|---|---|
| Fraction | Min. | Median | Max. | Mean | Dev. |
| a | 2.77 | 4.56 | 7.37 | 4.71778 | 1.33511 |
| (>0-20%) | | | | | |
| b | 2.65 | 5.78 | 12.99 | 6.40667 | 2.95100 |
| (>20-40%) | | | | | |
| c | 4.06 | 6.06 | 14.62 | 7.62667 | 3.52021 |
| (>40-60%) | | | | | |
| d | 2.81 | 6.08 | 19.93 | 8.29111 | 5.52633 |
| (>60-80%) | | | | | |
| e | 3.5 | 5.18 | 7.67 | 5.19556 | 1.36700 |
| (>80-100%) | | | | | |
| Full Breath | 2.78 | 4.47 | 11.19 | 5.48778 | 2.99063 |
| Online | 1.16 | 2.3 | 3.9 | 2.38000 | 0.76236 |

### Example 15

The results of breath analysis for breath compound 1-octene are shown in Table 15. The greatest median number of molecules was found in the first >60%-80% fraction of exhaled breath, the maximum number of molecules was found in the first >60%-80% fraction of the exhaled breath, and the greatest mean number of molecules was found in the first >60%-80% fraction of exhaled breath.

TABLE 15

| | | | | | Std. |
|---|---|---|---|---|---|
| Fraction | Min. | Median | Max. | Mean | Dev. |
| a | 11.39 | 15.26 | 31.36 | 19.2400 | 7.8315 |
| (>0-20%) | | | | | |
| b | 10.63 | 31.25 | 48.21 | 28.3256 | 14.5398 |
| (>20-40%) | | | | | |
| c | 16.95 | 32.59 | 47.88 | 33.1600 | 13.3729 |
| (>40-60%) | | | | | |
| d | 10.17 | 33.95 | 72.66 | 36.3211 | 21.4932 |
| (>60-80%) | | | | | |
| e | 7.97 | 31.38 | 36.53 | 25.4256 | 9.9956 |
| (>80-100%) | | | | | |
| Full Breath | 8.57 | 10.15 | 48.96 | 18.4911 | 14.3130 |
| Online | 5.3 | 7.11 | 9.3 | 7.2800 | 1.1963 |

### Example 16

The results of breath analysis for breath compound 3-methylhexane are shown in Table 16. The greatest median number of molecules was found in the first >40%-60% fraction of exhaled breath, the maximum number of molecules was found in the first >60%-80% fraction of the exhaled breath, and the greatest mean number of molecules was found in the first >60%-80% fraction of exhaled breath.

TABLE 16

| | | | | | Std. |
|---|---|---|---|---|---|
| Fraction | Min. | Median | Max. | Mean | Dev. |
| a | 10.71 | 15.19 | 41.19 | 19.4956 | 10.2175 |
| (>0-20%) | | | | | |
| b | 10.63 | 28.39 | 96.28 | 39.6767 | 29.3143 |
| (>20-40%) | | | | | |
| c | 20.91 | 55.93 | 93.1 | 53.3822 | 30.9687 |
| (>40-60%) | | | | | |
| d | 8.34 | 51.39 | 153.12 | 63.2444 | 49.4430 |
| (>60-80%) | | | | | |
| e | 10.5 | 30.62 | 56.12 | 32.9300 | 17.9731 |
| (>80-100%) | | | | | |
| Full Breath | 8.44 | 11.28 | 83.5 | 26.8667 | 27.8909 |
| Online | 5.5 | 6.6 | 9.22 | 6.8667 | 1.1378 |

### Example 17

The results of breath analysis for breath compound e-2-nonene are shown in Table 17. The greatest median number of molecules was found in the first >20%-40% fraction of exhaled breath, the maximum number of molecules was found in the first >60%-80% fraction of the exhaled breath, and the greatest mean number of molecules was found in the first >60%-80% fraction of exhaled breath.

TABLE 17

| | | | | | Std. |
|---|---|---|---|---|---|
| Fraction | Min. | Median | Max. | Mean | Dev. |
| a | 1.1 | 1.99 | 3.16 | 2.16667 | 0.66991 |
| (>0-20%) | | | | | |
| b | 1.25 | 2.67 | 3.16 | 2.56000 | 0.61364 |
| (>20-40%) | | | | | |
| c | 1.57 | 2.48 | 4.09 | 2.77889 | 0.91581 |
| (>40-60%) | | | | | |
| d | 1.5 | 2.62 | 4.91 | 2.85556 | 1.07073 |
| (>60-80%) | | | | | |
| e | 1.3 | 2.62 | 3.39 | 2.60778 | 0.59682 |
| (>80-100%) | | | | | |
| Full Breath | 1.27 | 1.66 | 3.52 | 2.16444 | 0.85307 |
| Online | 0.81 | 0.98 | 1.66 | 1.06222 | 0.23968 |

### Example 18

The results of breath analysis for breath compound ammonia are shown in Table 18. The greatest median number of molecules was found in the first >60%-80% fraction of exhaled breath, the maximum number of molecules was found in the first >60%-80% fraction of the exhaled breath, and the greatest mean number of molecules was found in the first >60%-80% fraction of exhaled breath.

TABLE 18

| | | | | | Std. |
|---|---|---|---|---|---|
| Fraction | Min. | Median | Max. | Mean | Dev. |
| a | 35.88 | 48.92 | 72.17 | 49.6511 | 11.2008 |
| (>0-20%) | | | | | |
| b | 31.34 | 47.12 | 71.48 | 49.5222 | 11.9845 |
| (>20-40%) | | | | | |
| c | 37.96 | 43.1 | 52.82 | 44.9933 | 5.6426 |
| (>40-60%) | | | | | |
| d | 33.99 | 49.81 | 59.85 | 46.6389 | 10.9525 |
| (>60-80%) | | | | | |
| e | 35.02 | 40.91 | 54.3 | 43.3889 | 6.6669 |
| (>80-100%) | | | | | |
| Full Breath | 34.38 | 59.94 | 98.78 | 60.9244 | 20.7928 |
| Online | 42.2 | 48.5 | 54.7 | 48.1000 | 3.8503 |

### Example 19

The results of breath analysis for breath compound ethane are shown in Table 19. The greatest median number of molecules was found in the first >60%-80% fraction of exhaled breath, the maximum number of molecules was found in the first >0%-20% fraction of the exhaled breath, and the greatest mean number of molecules was found in the first >0%-20% fraction of exhaled breath.

TABLE 19

| | | | | | Std. |
|---|---|---|---|---|---|
| Fraction | Min. | Median | Max. | Mean | Dev. |
| a | 63.48 | 66.75 | 73.22 | 67.9322 | 3.75889 |
| (>0-20%) | | | | | |
| b | 64.22 | 67.19 | 68.3 | 66.6856 | 1.48705 |
| (>20-40%) | | | | | |
| c | 64.21 | 66.73 | 69.98 | 67.2511 | 2.26901 |
| (>40-60%) | | | | | |
| d | 63.73 | 68.62 | 70.72 | 67.0433 | 2.64224 |
| (>60-80%) | | | | | |
| e | 62.49 | 67.71 | 71.66 | 67.7033 | 3.33546 |
| (>80-100%) | | | | | |
| Full Breath | 59.07 | 62.18 | 64.66 | 62.0511 | 1.62653 |
| Online | 64.7 | 66.8 | 70.5 | 67.0222 | 1.90183 |

### Example 20

The results of breath analysis for breath compound hydrogen sulfide are shown in Table 20. The greatest median number of molecules was found in the first >40%-60% fraction of exhaled breath, the maximum number of molecules was found in the first >40%-60% fraction of the exhaled breath, and the greatest mean number of molecules was found in the first >40%-60% fraction of exhaled breath.

TABLE 20

| | | | | | Std. |
|---|---|---|---|---|---|
| Fraction | Min. | Median | Max. | Mean | Dev. |
| a | 0.16 | 0.27 | 0.31 | 0.263333 | 0.048477 |
| (>0-20%) | | | | | |
| b | 0.15 | 0.24 | 0.52 | 0.253333 | 0.108513 |
| (>20-40%) | | | | | |
| c | 0.21 | 0.28 | 0.45 | 0.300000 | 0.068374 |
| (>40-60%) | | | | | |
| d | 0.19 | 0.28 | 0.43 | 0.287778 | 0.073617 |
| (>60-80%) | | | | | |
| e | 0.21 | 0.28 | 0.36 | 0.278889 | 0.043429 |
| (>80-100%) | | | | | |
| Full Breath | 0.17 | 0.28 | 0.58 | 0.305556 | 0.127290 |
| Online | 0.2 | 0.32 | 0.63 | 0.357778 | 0.132644 |

### Example 21

The results of breath analysis for breath compound triethyl amine are shown in Table 21. The greatest median number of molecules was found in the first >0%-20% fraction of exhaled breath, the maximum number of molecules was found in the first >60%-80% fraction of the exhaled breath, and the greatest mean number of molecules was found in the first >60%-80% fraction of exhaled breath.

TABLE 21

| | | | | | Std. |
|---|---|---|---|---|---|
| Fraction | Min. | Median | Max. | Mean | Dev. |
| a | 0.53 | 0.82 | 1.11 | 0.791111 | 0.176029 |
| (>0-20%) | | | | | |
| b | 0.5 | 0.81 | 1.31 | 0.862222 | 0.281681 |
| (>20-40%) | | | | | |
| c | 0.59 | 0.81 | 1.39 | 0.905556 | 0.276003 |
| (>40-60%) | | | | | |
| d | 0.7 | 0.91 | 1.62 | 0.982222 | 0.297396 |
| (>60-80%) | | | | | |
| e | 0.61 | 0.81 | 1.13 | 0.816667 | 0.180762 |
| (>80-100%) | | | | | |
| Full Breath | 0.5 | 0.62 | 1.12 | 0.722222 | 0.233440 |
| Online | 0.27 | 0.44 | 1.12 | 0.563333 | 0.307612 |

### Example 22

The results of breath analysis for breath compound trimethyl amine are shown in Table 22. The greatest median number of molecules was found in the first >40%-60% fraction of exhaled breath, the maximum number of molecules was found in the first >80%-100% fraction of the exhaled breath, and the greatest mean number of molecules was found in the first >40%-60% fraction of exhaled breath.

TABLE 22

| | | | | | Std. |
|---|---|---|---|---|---|
| Fraction | Min. | Median | Max. | Mean | Dev. |
| a | 1.07 | 2.94 | 10.36 | 3.7811 | 2.86532 |
| (>0-20%) | | | | | |
| b | 2.02 | 2.98 | 6.43 | 3.5789 | 1.38418 |
| (>20-40%) | | | | | |
| c | 2.58 | 5.73 | 11.96 | 5.9322 | 3.19743 |
| (>40-60%) | | | | | |
| d | 1.21 | 4.52 | 12.45 | 5.6778 | 3.50038 |
| (>60-80%) | | | | | |
| e | 1.16 | 3.4 | 15.26 | 4.2556 | 4.33017 |
| (>80-100%) | | | | | |
| Full Breath | 1.45 | 3.02 | 22.28 | 5.2400 | 6.68710 |
| Online | 5.5 | 8.6 | 30 | 11.3422 | 7.66736 |

A summary of the various breath fractions that had elevated ranges of molecules, (i.e. median concentration, maximum concentration, and mean concentration) for each of the volatile organic compounds in Examples 1-22 above is listed in Table 23.

TABLE 23

| | Median | Maximum | Mean |
|---|---|---|---|
| | Concentration | Concentration | Concentration |
| | (Breath | (Breath | (Breath |
| Compound | Fraction) | Fraction) | Fraction) |
| 2-Propanol | >60-80% | >40-60% | >60-80% |
| Acetaldehyde | >60-80% | >40-60% | >40-60% |
| Acetone | >40-60% | >0-20% | >40-60% |
| Acetonitrile | >40-60% | >60-80% | >60-80% |
| Acrylonitrile | >60-80% | >40-60% | >60-80% |
| Benzene | >40-60% | >40-60% | >40-60% |
| Carbon Disulfide | >20-40% | >40-60% | >40-60% |
| Dimethyl Sulfide | >80-100% | >60-80% | >40-60% |
| Ethanol | >40-60% | >20-40% | >20-40% |
| Isoprene | >40-60% | >60-80% | >40-60% |
| Pentane | >60-80% | >40-60% | >60-80% |
| 1-Decene | >60-80% | >60-80% | >60-80% |
| 1-Heptene | >60-80% | >60-80% | >60-80% |
| 1-Nonene | >60-80% | >60-80% | >60-80% |
| 1-Octene | >60-80% | >60-80% | >60-80% |
| 3-Methylhexane | >40-60% | >60-80% | >60-80% |
| E-2-Nonene | >20-40% | >60-80% | >60-80% |
| Ammonia | >60-80% | >60-80% | >60-80% |
| Ethane | >60-80% | >0-20% | >0-20% |
| Hydrogen Sulfide | >40-60% | >40-60% | >40-60% |
| Triethyl Amine | >0-20% | >60-80% | >60-80% |
| Trimethyl Amine | >40-60% | >80-100 | >40-60% |

The complete disclosure of all patents, patent applications, and publications, and electronically available material cited herein are incorporated by reference. The foregoing detailed description and examples have been given for clarity of understanding only. No unnecessary limitations are to be understood therefrom. Although the invention has been described with reference to several specific embodiments, the invention is not limited to the exact details shown and described, for variations obvious to one skilled in the art will be included. The description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

What is claimed is:

1. A method of collecting breath from a subject, the method comprising:
capturing a target sample from the non-alveolar volume fraction of an exhaled breath of a subject, the target sample having a target concentration of a target volatile organic compound (VOC) in the exhaled breath, wherein the exhaled breath represents the lung capacity of the subject.

2. The method of claim 1, comprising determining the lung capacity of the subject prior to capturing the target sample.

3. The method of claim 1, wherein the target sample having an target concentration of a volatile organic compound (VOC) is captured in the first about 80% volume fraction of the exhaled breath stream.

4. The method of claim 1, wherein the target sample is captured in the about 20% to about 80% volume fraction of the exhaled breath stream.

5. The method of claim 1, wherein the exhaled breath stream has a volumetric flow rate that ranges from about 250 mL/sec to about 500 mL/sec.

6. The method of claim 1, wherein the exhaled breath stream has a volumetric flow rate that ranges from about 350 mL/sec to about 450 mL/sec.

7. The method of claim 1, wherein the target VOC compound is selected from the group consisting of: nitric oxide, isoprene, beta hydroxybutyrate, 2-propanol, acetaldehyde, acetone, acetonitrile, acrylonitrile, benzene, carbon disulfide, dimethyl sulfide, ethanol, isoprene, pentane, 1-decene, 1-heptane, 1-nonene, 1-octene, 3-methylhexane, E-2-nonene, ammonia, ethane, hydrogen sulfide, triethyl amine, trimethyl amine.

8. The method of claim 1, wherein the target sample is captured in the first about 60% volume fraction of the exhaled breath stream.

9. The method of claim 8, wherein the target concentration is an elevated concentration and the target VOC compound is selected from the group consisting of: 2-propanol, acetaldehyde, acetone, acetonitrile, acrylonitrile, benzene, carbon disulfide, dimethyl sulfide, ethanol, isoprene, pentane, 3-methylhexane, E-2-nonene, ethane, hydrogen sulfide, triethyl amine, and trimethyl amine.

10. The method of claim 1, wherein the target sample is collected in the first about 40% to about 80% volume fraction of the exhaled breath stream.

11. The method of claim 10, wherein the target concentration is an elevated concentration and the preselected breath compound is selected from the group of: 2-propanol, acetaldehyde, acetonitrile, acrylonitrile, benzene, carbon disulfide, dimethyl sulfide, ethanol, isoprene, pentane, 1-decene, 1-heptane, 1-nonene, 1-octene, 3-methylhexane, e-2-nonene, ammonia, ethane, hydrogen sulfide, triethyl amine, and trimethyl amine.

12. The method of claim 1, wherein the target sample represents from about 5% to about 25% by volume of the exhaled breath.

13. The method of claim 1, wherein the target sample represents up to about 20% by volume of the exhaled breath.

14. The method of claim 1, wherein the target sample comprises an target concentration of a second target volatile organic compound (VOC), the second target VOC being a different volatile organic compound (VOC) from the target volatile organic compound (VOC).

15. The method of claim 1, comprising:
capturing a second target sample comprising an target concentration of a second target volatile organic compound (VOC) in the exhaled breath; and
wherein the second target sample is captured from the first about 80% volume fraction of the exhaled breath and is a different volume fraction of the exhaled breath than the target sample of the exhaled breath.

16. The method of claim 1, further comprising rinsing the mouth of a subject before capturing the target sample.

17. The method of claim 1, wherein the exhaled breath is filtered.

18. The method of claim 1, wherein the subject inhales air through a filter before exhaling the exhaled breath.

19. The method of claim 1, wherein target sample is captured from the first about 70% volume fraction of the exhaled breath stream.

20. A non-transitory, machine-readable medium having machine-executable instructions configured to:
capture a target sample in a non-alveolar volume fraction of an exhaled breath of a subject, the target sample comprising an target concentration of a target volatile organic compound; and
wherein the exhaled breath represents the lung capacity of the subject.

21. The non-transitory, machine-readable medium of claim 20, wherein the target sample comprises an target concentration of a second target volatile organic compound (VOC), wherein the second target volatile organic compound (VOC) is different than the target volatile organic compound (VOC).

22. The non-transitory, machine-readable medium of claim 20, the machine-executable instructions further configured to:
capture a second target sample in the non-alveolar volume fraction of the exhaled breath of a subject, the second target sample comprising an target concentration of a second target volatile organic compound, and the second target sample is a different fraction than the target sample of the exhaled breath.

23. The non-transitory, machine-readable medium of claim 20, the machine-executable instructions further configured to:
receive exhaled breath data that characterize an exhaled breath of a patient; and
capture the target sample based on the exhaled breath data.

24. The non-transitory, machine-readable medium of claim 23, wherein exhaled breath data include a volumetric flow rate of the exhaled breath that ranges from about 250 mL/sec to about 500 mL/sec.

25. The non-transitory, machine-readable medium of claim 23, wherein the exhaled breath data comprise information relating to at least one of exhaled breath velocity, time, lung capacity, pressure, temperature and humidity.

26. The non-transitory, machine-readable medium of claim 20, wherein the target volatile organic compound is selected from the group consisting of: 2-propanol, acetaldehyde, acetone, acetonitrile, acrylonitrile, benzene, disulfide, dimethyl sulfide, ethanol, isoprene, pentane, 1-decene, 1-heptane, 1-nonene, 1-octene, 3-methylhexane, E-2-nonene, ammonia, ethane, hydrogen sulfide, triethyl amine, and trimethyl amine.

27. A system comprising:
a breath collection device; and
a computing device in communications with the breath collection device, the computing device comprising non-transitory, machine-readable medium having machine-executable instructions configured to:
capture a target sample in a non-alveolar volume fraction of an exhaled breath of a subject, the target sample having an target concentration of a target volatile organic compound; and
wherein the exhaled breath represents substantially the lung capacity of the subject.

28. The breath collection system of claim 27, wherein the system comprises an analytical device.

29. The breath collection system of claim 28, wherein the analytical device is a sensor.

30. The breath collection system of claim 27, wherein the target volatile organic compound is selected from the group consisting of: nitric oxide, isoprene, beta hydroxybutyrate, 2-propanol, acetaldehyde, acetone, acetonitrile, acrylonitrile, benzene, disulfide, dimethyl sulfide, ethanol, isoprene, pentane, 1-decene, 1-heptane, 1-nonene, 1-octene, 3-methylhexane, E-2-nonene, ammonia, ethane, hydrogen sulfide, triethyl amine, and trimethyl amine.
